(19) United States
(12) Patent Application Publication
(10) Pub. No.: US 2004/0215299 A1
(43) Pub. Date: Oct. 28, 2004

(54) Implantable medical device conductor insulation and process for forming

(75) Inventors: Yong David Zhao, Plymouth, MN (US); John L. Sommer, Coon Rapids, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/421,182

(22) Filed: Apr. 23, 2003

(51) Int. Cl. A61N 1/05

(52) U.S. Cl. 607/116; 607/122

(57) ABSTRACT

A composite redundant insulation is formed about each of a plurality of conductors extending within a lead body of an implantable medical device. The insulation includes a first insulative layer, a second insulative layer having a lower durometer and a lower flexural modulus than the first insulative layer, and a third insulative layer having a higher durometer and a higher flexural modulus than the second insulative layer.

### RELATED APPLICATION

This application is a continuation-in-part of U.S. patent application Ser. No. ______ (Attorney docket P10722.00) filed on Apr. 4, 2003 and entitled “IMPLANTABLE MEDICAL DEVICE CONDUCTOR INSULATION AND PROCESS FOR FORMING”, which claims priority and other benefits from U.S. Provisional Patent Application Serial No. 60/371,995, filed Apr. 11, 2002, entitled “BIO-STABLE IMPLANTABLE MEDICAL DEVICE LEAD CONDUCTOR INSULATION AND PROCESS FOR FORMING”, both of which are incorporated herein by reference in their entireties.

### FIELD OF THE INVENTION

The present invention relates generally to implantable medical device leads for delivering therapy, in the form of electrical stimulation, and in particular, the present invention relates to conductor coil insulation in implantable medical device leads.

### BACKGROUND OF THE INVENTION

Implantable medical electrical leads are well known in the fields of cardiac stimulation and monitoring, including neurological pacing and cardiac pacing and cardioversion/defibrillation. In the field of cardiac stimulation and monitoring, endocardial leads are placed through a transvenous route to position one or more sensing and/or stimulation electrodes in a desired location within a heart chamber or interconnecting vasculature. During this type of procedure, a lead is passed through the subclavian, jugular, or cephalic vein, into the superior vena cava, and finally into a chamber of the heart or the associated vascular system. An active or passive fixation mechanism at the distal end of the endocardial lead may be deployed to maintain the distal end of the lead at a desired location.

Routing an endocardial lead along a desired path to a target implant site can be difficult and is dependent upon the physical characteristics of the lead. At the same time, as will be readily appreciated by those skilled in the art, it is highly desirable that the implantable medical lead insulation possess high dielelectric properties, and exhibit durable and bio-stable properties, flexibility, and reduced size.

One type of lead includes a body formed, in part by a plurality of conductive wires formed in a coil. Each of a plurality of electrodes, formed about a distal portion of the lead, is electrically coupled to each of a plurality of electrical contacts, formed about a proximal portion of the lead, by one or a group of the plurality of conductive wires; each wire or group of wires coupled to each electrode must be electrically isolated from one another. An insulation formed around the wires for electrical isolation must have sufficient dielectric strength, biostability and durability while maintaining a minimum thickness.

### BRIEF DESCRIPTION OF THE DRAWINGS

Embodiments of the present invention will be readily appreciated as the same becomes better understood by reference to the following detailed description when considered in connection with the accompanying drawings, in which like reference numerals designate like parts throughout the figures thereof and wherein:

FIG. 1 is a schematic diagram of an exemplary implantable medical device in accordance with the present invention;

FIG. 2 is a cross-sectional view of a lead of an implantable medical device according to the present invention, taken along cross-sectional lines II-II of FIG. 1;

FIG. 3 is a cross-sectional view of a lead of an implantable medical device according to the present invention, taken along cross-sectional lines III-III of FIG. 1;

FIG. 4 is a cross-sectional view of a coiled wire conductor forming a multi-filar conductor coil according to an embodiment of the present invention;

FIG. 5 is a cross-sectional view of a coiled wire conductor forming a multi-filar conductor coil according to another embodiment of the present invention;

FIG. 6 is a cross-sectional view of a coiled wire conductor forming a multi-filar conductor coil according to yet another embodiment of the present invention; and

FIG. 7 is a schematic illustrating a cantilever coil model used in a Finite Element Analysis of embodiments of the present invention.

### DETAILED DESCRIPTION OF THE INVENTION

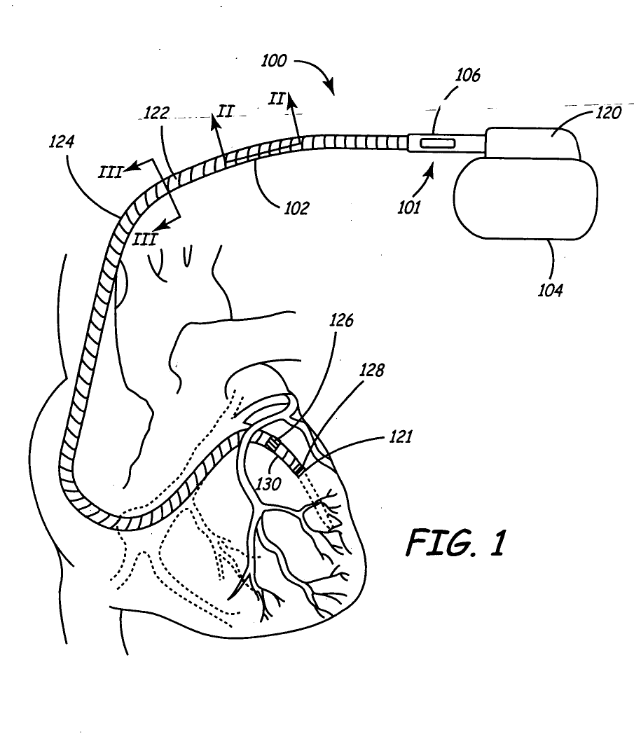

FIG. 1 is a schematic diagram of an exemplary implantable medical device in accordance with the present invention. As illustrated in FIG. 1, an implantable medical device 100 according to the present invention includes an implantable medical device lead 102 and an implantable medical device housing 104, such as an implantable cardioverter/defibrillator or pacemaker/cardioverter/defibrillator (PCD), for example, for processing cardiac data sensed through lead 102 and generating electrical signals in response to the sensed cardiac data for the provision of cardiac pacing, cardioversion and defibrillation therapies. A connector assembly 106 located at a proximal end 101 of lead 102 is insertable within a connector block 120 of housing 104 to electrically couple lead 102 with electronic circuitry (not shown) of housing 104.

Lead 102 includes an elongated lead body 122 that extends between proximal end 101 and a distal end 121 of lead 102. An outer insulative sheath 124 surrounds lead body 122 and is preferably fabricated of polyurethane, silicone rubber, or an ethylene tetrafluoroethylene (ETFE) or a polytetrafluoroethylene (PTFE) type coating layer. Coiled wire conductors in accordance with the present invention are positioned within lead body 122, as will be described in detail below. Distal end 121 of lead 102 includes a proximal ring electrode 126 and a distal tip electrode 128, separated by an insulative sleeve 130. Proximal ring electrode 126 and distal tip electrode 128 are electrically coupled to connector assembly 106 by one or more coil conductors, or filars extending between distal end 121 and proximal end 101 of lead 102 in a manner shown, for example, in U.S. Pat. Nos. 4,922,607 and 5,007,435, incorporated herein by reference in their entireties.

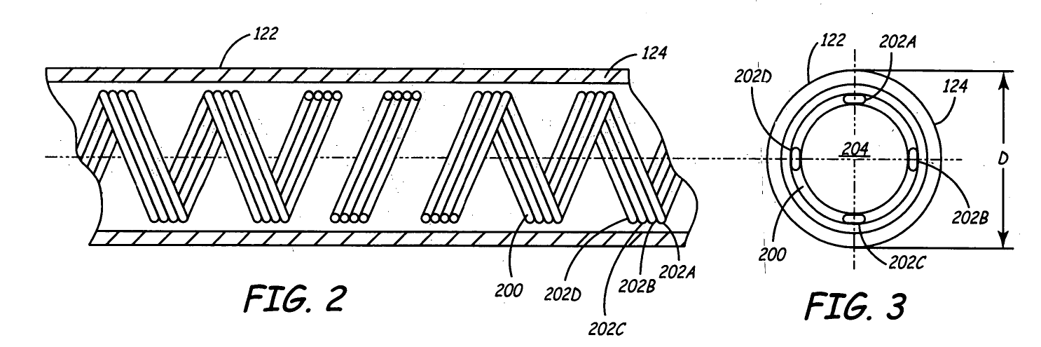

FIG. 2 is a cross-sectional view of a lead of an implantable medical device according to the present invention, taken along cross-sectional lines II-II of FIG. 1. As illustrated in FIG. 2, lead 102 of implantable medical device 100 includes a quadrifilar conductor coil 200 including four individual filars, or coiled wire conductors 202A, 202B, 202C and 202D extending within insulative sheath 124 of lead body 122. Coiled wire conductors 202A-202D electrically couple proximal ring electrode 126 and distal tip electrode 128 with connector assembly 106. It is understood that although the present invention is described throughout in the context of a quadrafilar conductor coil, having each of two electrodes electrically coupled to a connector assembly via two of the four individual coiled wire conductors, the present invention is not intended to be limit to application in a quadrafilar conductor coil. Rather, the lead conductor insulator of the present invention can be utilized in any conductor configuration, including the use of any number of conductor coils depending upon the number of desired electrodes, and would include the use of a single filar electrically coupling the electrode to the connector.

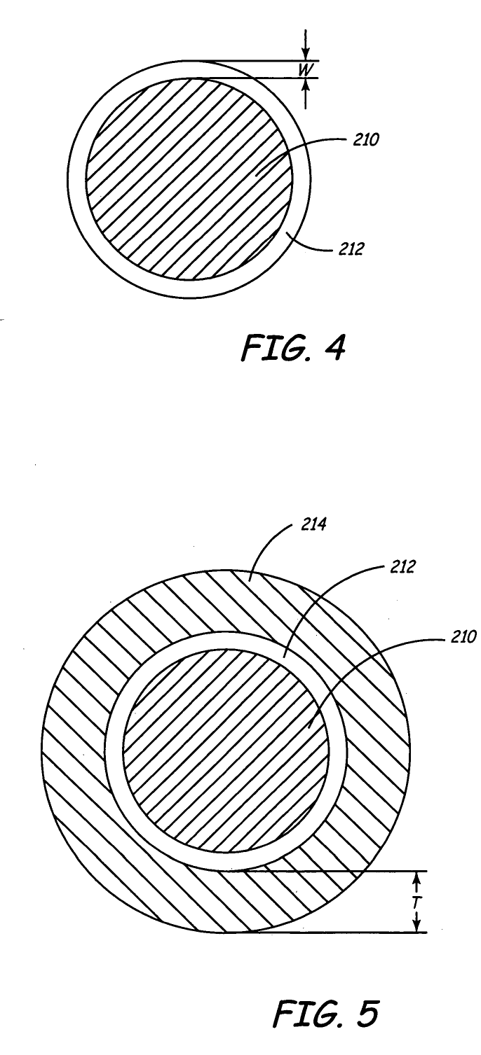

FIG. 3 is a cross-sectional view of a lead of an implantable medical device according to the present invention, taken along cross-sectional lines III-III of FIG. 1. As illustrated in FIGS. 2 and 3, each of the individual filars or coiled wire conductors 202A, 202B, 202C and 202D are parallel-wound in an interlaced manner to have a common outer and inner coil diameter. As a result, conductor coil 200 forms an internal lumen 204, which allows for passage of a stylet or guide wire (not shown) within lead 102 to direct insertion of lead 102 within the patient.

Alternately, lumen 204 may house an insulative fiber, such as ultrahigh molecular weight polyethylene (UHMWPE), liquid crystal polymer (LCP) and so forth, or an insulated cable in order to allow incorporation of an additional conductive circuit and/or structural member to aid in chronic removal of lead 102 using traction forces. Such an alternate embodiment would require insertion and delivery of lead 102 to a final implant location using alternate means, such as a catheter, for example. Lumen 204 may also include an insulative liner (not shown), such as a fluoropolymer, polyimide, PEEK, for example, to prevent damage caused from insertion of a style/guidewire (not shown) through lumen 204.

FIG. 4 is a cross-sectional view of a coiled wire conductor forming a multi-filar conductor coil according to one embodiment of the present invention. As illustrated in FIG. 4, one or more of the individual coiled wire conductors 202A, 202B, 202C and 202D includes a conductor wire 210 surrounded by an insulative layer 212. According to the present invention, insulative layer 212 is formed of a hydrolytically stable polyimide, such as a Soluble Imide (SI) polyimide material, for example, (formerly known as Genymer, Genymer SI, and LARC SI) as described in U.S. Pat. No. 5,639,850, issued to Bryant, and incorporated herein by reference in it's entirety, to insulate conductor coils in implantable medical device leads. Such SI polyimide material is currently commercially available from Dominion Energy, Inc. (formerly Virginia Power Nuclear Services), for example. The thickness of the insulative layer 212 ranges from approximately 0.0001 inches up to approximately 0.0050 inches, forming a corresponding wall thickness W of the insulative layer 212. By utilizing the hydrolytically stable polyimide material as an insulative layer 212, the present invention provides an improved electrically insulating material that is hydrolytically stable in implantable (in vivo) applications. Furthermore, the use of a thin layer of hydrolytically stable polyimide coating on conventional MP35N alloy coil filars will also act as a protective barrier to reduce the incidence of metal induced oxidation seen on some polyurethane medical device insulations.

According to the present invention, the insulative layer 212 is applied onto the conductor wire 210 in multiple coats to obtain a desired wall thickness W. The coating is applied in such a way to provide a ductile, robust insulative layer that enables a single filar, i.e., coiled wire conductor, or multiple filar, i.e., coiled wire conductors, to be wound into a single wound conductor coil 200 of sizes ranging from an outer diameter D (FIG. 3) of 0.010 inches to 0.110 inches. For example, according to the present invention, the coating process includes a solvent dip followed by an oven cure cycle to drive off the solvents. The multiple coating passes during the application of the insulative layer 212 onto the conductor wire 210 provides the ductility between layers that is needed to make the coated conductor wire 210 into a very tight wound conductor coil 200 and that can withstand the long term flex requirements of an implantable stimulating lead. As a result, the material is hydrolytically stable over time, and the process of applying the SI polyimide in thin coatings, through multiple passes, provides a ductile polyimide that can be wound into a conductor coil.

The use of the hydrolytically stable polyimide insulative layer 212 according to the present invention offers an exceptional dielectric strength and provides electrical insulation. Through flex studies on conductor coils coated with the SI polyimide, for example, the inventors have found that the insulative layer 212 also has high flex properties in regards to stimulating lead conductor coil flex testing. The SI coating in various wall thicknesses will remain intact on the coil filar until the coil filar fractures as seen in conventional conductor coil flex studies (reference 10 million to 400 million flex cycles at various 90 degree radius bends).

Conductor coils 200 (FIG. 2) according to the present invention, can include a single filar or multiple filars, with each filar being an individual circuit that could be associated with either a tip electrode, a ring electrode, a sensor, and so forth. In known lead designs, each lead utilizes one coil per circuit with a layer of insulation. The present invention enables the use of multiple circuits in a single conductor coil, resulting in a downsizing of the implantable medical device. For example, there is approximately a 40 to 50 percent reduction in lead size between known bipolar designs, which traditionally utilized an inner coil and inner insulation, outer coil and outer insulation, to a lead design having multiple circuits in a single conductor coil having the insulative layer 212 according to the present invention.

FIG. 5 is a cross-sectional view of a coiled wire conductor forming a multi-filar conductor coil according to another embodiment of the present invention. The insulative layer 212 of the present invention can be utilized as a stand-alone insulation on a filar or as an initial layer of insulation followed by an additional outer layer as redundant insulation to enhance reliability. For example, according to an embodiment of the present invention illustrated in FIG. 5, in addition to conductor wire 210 and insulative layer 212, one or more of the individual coiled wire conductors 202A, 202B, 202C and 202D includes an additional outer insulative layer 214, formed of known insulative materials, such as ETFE, for example, to enhance reliability of the lead. According to the present invention, insulative layer 214 generally has a thickness T between approximately 0.0005 and 0.0025 inches, for example, although other thickness ranges are contemplated by the present invention. Since the outermost insulative layer, i.e., insulative layer 214, experiences more displacement during flex of lead 102 than insulative layer 212, it is desirable for insulative layer 214 to be formed of a lower flex modulus material than insulative layer 212, such as ETFE.

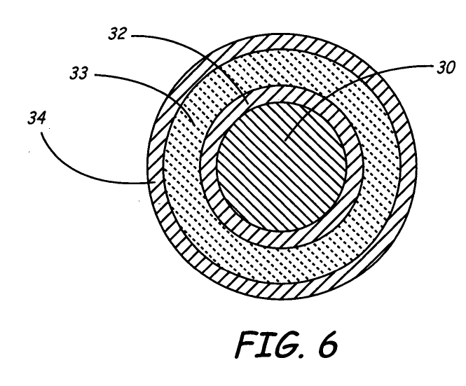

FIG. 6 is a cross-sectional view of a coiled wire conductor forming a multi-filar conductor coil according to yet another embodiment of the present invention. FIG. 6 illustrates a composite redundant insulation formed about a conductor wire 30 and including a first insulative layer 32, a second insulative layer 33 and a third insulative layer 34. Conductor wire 30 forms one or more of individual coiled wire conductors, for example coil wire conductors 202A, 202B, 202C, and 202D illustrated in FIGS. 2 and 3, and has a diameter between approximately 0.0008 inch and 0.005 inch. According to embodiments of the present invention, layers 32, 33, and 34 function synergistically to preserve electrical isolation between individual coiled wires of a lead body, for example lead body 122 illustrated in FIGS. 1-3, as the lead body is subjected to tension, compression, bending and torsion loads of an implant environment such as that illustrated in FIG. 1. Furthermore the composite construction provides enhanced durability under coil winding loads.

First insulative layer 32 corresponds to previously described insulative layer 212, being a hydrolytically stable polyimide, such as the Soluble Imide (SI) polyimide material referenced above. As previously described for layer 212, first layer 32 is applied in thin coatings through multiple passes. Second layer 33, having a thickness between approximately 0.0005 inch and 0.003 inch, is formed of a material having a lower flexural modulus and durometer or hardness than first layer 32; suitable materials include polyurethanes and fluoropolymers, for example ETFE or PTFE. Third layer 34, having a thickness between approximately 0.0005 inch and 0.002 inch, is formed of a material having a higher flexural modulus and durometer or hardness than second insulative layer 33; suitable materials include polyurethanes and fluoropolymers. According to embodiments of the present invention, a combination of first insulative layer 32, second insulative layer 33 and third insulative layer 34 provides improved redundancy while maintaining a minimum overall insulation thickness within a range of approximately 0.002 inch to 0.005 inch. Second insulative layer 33 provides insulation redundancy by filling or covering any voids or thin zones in first layer 32 and second insulative layer 32 is protected from wear by third insulative layer 34; the combination of layers allows first insulative layer 32 to be thinner, on average, than previously described layer 212, thus requiring fewer thin coating passes to form first layer 32; for example, a thickness of layer 32 between approximately 0.0001 inch and 0.001 inch. Further, second insulative layer 33 acts as an impact absorber between first insulative layer 32 and third insulative layer 34. According to some embodiments of the present invention, interfacing surfaces of each layer 32, 33, and 34 are not bonded to one another and, in a subset of embodiments, each layer 32, 33, and 34 is free to slide one against the other increasing a ductility and flexibility of the composite insulation.

According to embodiments of the present invention, second insulative layer 33 is formed about first insulative layer 32 and then third insulative layer 34 is formed about second insulative layer 33, each with minimal clearance approaching zero; co-extrusion processes, known to those skilled in the art, may be used to form layers 33 and 34. Suitable materials for second insulative layer 33 and third insulative layer 34, according to some embodiments, have melt temperatures that facilitate co-extrusion while preventing bonding of second layer 33 to first layer 32 and third layer 34 to second layer 33, for example, second layer 33 has a melt temperature between approximately 400° F. and 500° F. and third layer has a melt temperature less than approximately 400° F., while first layer 32 has a melt temperature between approximately 500° F. and 750° F.

### EXAMPLE

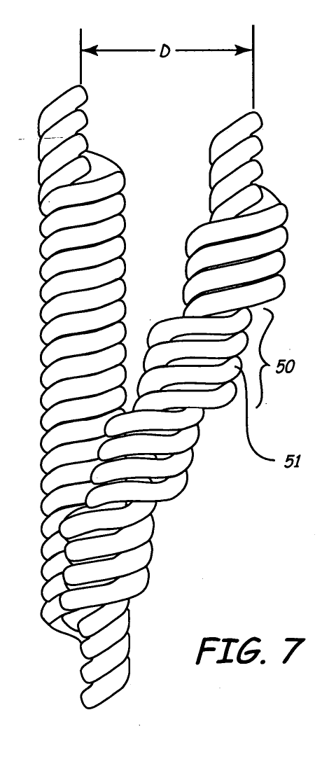

As an illustrative example, a finite element analysis (FEA) was completed to calculate maximum principal strains of insulation formed about filars of cantilever coils including four filars under single prescribed tension, bending, compression and torsion displacement loading. Five turns of each coil were modeled, including all possible physical contact interactions between filars, and maximum principal strain contours were generated for an insulative layer of a second filar at a central segment of each coil model. FIG. 7 illustrates a cantilever coil model including four filars and five turns separately subjected to a single bending load of a prescribed displacement, approximated by “D”, wherein a maximum principal strain contour was generated for a second filar 51 of a central segment 50. Each coil model included filars having a diameter equal to 0.0035 inch and approximately the mechanical properties of MP35N high strength alloy. Furthermore, a total insulation thickness for filars of each coil model was 0.0020 inch. A filar insulation of a first coil model included one layer having approximately the mechanical properties of SI polyimide, while a filar insulation of a second coil model included three insulative layers: a first layer having approximately the mechanical properties of SI polyimide material, a second layer having approximately the mechanical properties of ETFE, and a third layer having mechanical properties corresponding to a material having a greater hardness than that of the second layer. A cross-section of a filar of the second coil model is generally represented in FIG. 6. In the FEA material models input, each material of the insulation was modeled using elastic-plastic models from empirically derived stress-strain curves. Table 1 presents a thickness and Young's modulus, or flexural modulus, for each insulative layer of each coil model.

TABLE 1
Coil Model properties

| | 1^{st} coil model, | | |
|---|---|---|---|
| | one insulative | 2^{nd} coil model, | |
| | layer | three insulative layers | |
| Thickness, | 0.0020 | 1^{st} layer: | 0.0005 |
| inch | | 2^{nd} layer: | 0.0010 |
| | | 3^{rd} layer: | 0.0005 |
| Young's | 414,136 | 1^{st} layer: | 414,136 |
| Modulus, psi | | 2^{nd} layer: | 69,255 |
| | | 3^{rd} layer: | 83,106 |

In the second coil model, obvious discontinuous strain contours in a radial direction were observed, and strain gradients were significantly reduced. The maximum principal strain in the first insulative layer of the second coil model was reduced by 39.81% under tension, by 40.75% under compression, by 50.55% under bending, and by 29.46% under torsion over that of the one insulative layer of the first coil model. Table 2 presents the maximum principal strain (in/in) in the one layer of the first coil model versus the first layer of the second coil model for each type of load.

TABLE 2
Maximum principal strains (in/in)

| | 1^{st} coil model, | 2^{nd} coil model, |
|---|---|---|
| | one insulative | three insulative |
| | layer | layers |
| Tension load | 0.03665 | 0.02206 |
| Compression | 0.01392 | 0.008248 |
| load | | |
| Bending load | 0.00156 | 0.0007714 |
| Torsion load | 0.007891 | 0.005566 |

While particular embodiments of the present invention have been shown and described, modifications may be made. It is therefore intended in the appended claims to cover all such changes and modifications, which fall within the true spirit and scope of the invention.

1. An implantable medical device, comprising:
a lead body extending from a proximal end to a distal end;
a plurality of conductors extending within the lead body from the proximal end to the distal end; and
a composite redundant insulation formed about each of the plurality of conductors and including a first insulative layer, a second insulative layer having a lower durometer and a lower flexural modulus than the first insulative layer, and a third insulative layer having a higher durometer and a higher flexural modulus than the second insulative layer.

2. The implantable medical device of claim 1, wherein the first insulative layer comprises a hydrolytically stable polyimide material.

3. The implantable medical device of claim 2, wherein the hydrolytically stable polyimide material is an SI polyimide.

4. The implantable medical device of claim 1, wherein the second insulative layer comprises a fluoropolymer.

5. The implantable medical device of claim 4, wherein the fluoropolymer is an ETFE.

6. The implantable medical device of claim 1, wherein the second insulative layer comprises polyurethane.

7. The implantable medical device of claim 1, wherein the third insulative layer comprises a fluoropolymer.

8. The implantable medical device of claim 1, wherein the third insulative layer comprises polyurethane.

9. The implantable medical device of claim 1, wherein the first insulative layer has a thickness between approximately 0.0001 inch and approximately 0.001 inch.

10. The implantable medical device of claim 1, wherein the composite redundant insulation has a thickness between approximately 0.002 inch and approximately 0.005 inch.

11. The implantable medical device of claim 1, wherein the second layer is free to slide against the first layer and the third layer is free to slide against the second layer.

12. An implantable medical device, comprising:
a lead body extending from a proximal end to a distal end;
a plurality of conductors extending within the lead body from the proximal end to the distal end;
a first insulative layer formed about each of the plurality of conductors by a dip coating process and comprised of a hydrolytically stable polyimide;
a second insulative layer formed about the first insulative layer by a co-extrusion process and having a lower durometer and a lower flexural modulus than the first insulative layer; and
a third insulative layer formed about the second insulative layer by a co-extrusion process and having a higher durometer and a higher flexural modulus than the second insulative layer.

13. The implantable medical device of claim 12, wherein the hydrolytically stable polyimide material is an SI polyimide.

14. The implantable medical device of claim 12, wherein the second insulative layer comprises a fluoropolymer.

15. The implantable medical device of claim 14, wherein the fluoropolymer is an ETFE.

16. The implantable medical device of claim 12, wherein the second insulative layer comprises polyurethane.

17. The implantable medical device of claim 12, wherein the third insulative layer comprises a fluoropolymer.

18. The implantable medical device of claim 12, wherein the third insulative layer comprises polyurethane.

19. The implantable medical device of claim 12, wherein the first insulative layer has a thickness between approximately 0.0001 inch and approximately 0.001 inch.

20. The implantable medical device of claim 12, wherein the composite redundant insulation has a thickness between approximately 0.002 inch and approximately 0.005 inch.

21. The implantable medical device of claim 12, wherein the second layer is free to slide against the first layer and the third layer is free to slide against the second layer.

22. The implantable medical device of claim 12, wherein the second layer has a melt temperature between approximately 400° F. and approximately 500° F.; and the third insulative layer has melt temperature less than approximately 400° F.

23. An implantable medical device, comprising:
a housing adapted to generate electrical signals and including a connector block;
a lead body extending from a proximal end to a distal end and including a connector assembly terminating the proximal end and adapted to be coupled to the connector block of the housing;
a plurality of conductors coupled to the connector assembly and extending within the lead body, the plurality of conductors adapted to deliver the electrical signals from the housing, via the connector assembly, to an implant site; and
a composite redundant insulation formed about each of the plurality of conductors and including a first insulative layer, a second insulative layer having a lower durometer and a lower flexural modulus than the first insulative layer, and a third insulative layer having a higher durometer and a higher flexural modulus than the second insulative layer.

24. The implantable medical device of claim 23, wherein the first insulative layer comprises a hydrolytically stable polyimide material.

25. The implantable medical device of claim 23, wherein the hydrolytically stable polyimide material is an SI polyimide.

26. The implantable medical device of claim 23, wherein the second insulative layer comprises a fluoropolymer.

27. The implantable medical device of claim 26, wherein the fluoropolymer is an ETFE.

28. The implantable medical device of claim 23, wherein the second insulative layer comprises polyurethane.

29. The implantable medical device of claim 23, wherein the third insulative layer comprises a fluoropolymer.

30. The implantable medical device of claim 23, wherein the third insulative layer comprises polyurethane.

31. The implantable medical device of claim 23, wherein the first insulative layer has a thickness between approximately 0.0001 inch and approximately 0.001 inch.

32. The implantable medical device of claim 23, wherein the composite redundant insulation has a thickness between approximately 0.002 inch and approximately 0.005 inch.

33. The implantable medical device of claim 23, wherein the second layer is free to slide against the first layer and the third layer is free to slide against the second layer.
